(19) United States
(12) Patent Application Publication
(10) Pub. No.: US 2017/0328916 A1
(43) Pub. Date: Nov. 16, 2017

(54) RAPID DIAGNOSTIC KIT FOR CENTRAL NERVOUS SYSTEM AFFLICTIONS AND TRAUMATIC BRAIN INJURIES

(71) Applicant: Applied Nuerologics, LLC, W. Bloomfield, MI (US)

(72) Inventors: Mark Wasserman, Detroit, MI (US); Todd Flower, W. Bloomfield, MI (US); David Matzilevich, W. Bloomfield, MI (US)

(21) Appl. No.: 15/214,443

(22) Filed: Jul. 19, 2016

Related U.S. Application Data
(63) Continuation of application No. 15/152,193, filed on May 11, 2016.

(51) Int. Cl. G01N 33/68; G01N 33/543; G01N 33/58

(52) U.S. Cl. CPC G01N 33/6896; G01N 33/587; G01N 33/54386; G01N 33/585

(57) ABSTRACT

A method and system for diagnosing concussions, TBI, CTE and other central nervous system disorders is provided. A diagnostic kit includes a tube containing a predefined amount of lyophilized biomarker-specific antibody conjugated to a conjugate with a color functionality, a fluid for mixing with the lyophilized biomarker-specific antibody conjugated to the conjugate within the tube, so as to reconstitute the lyophilized biomarker-specific antibody conjugated to the conjugate, resulting in a reconstituted mixture, and a swab comprising an absorbent hydrophobic material, the swab configured for absorbing cerebrospinal fluid when swabbed on a patient, wherein when the swab that has absorbed cerebrospinal fluid is placed in the tube containing the reconstituted mixture, the swab is configured to change color.

### CROSS-REFERENCE TO RELATED APPLICATIONS

This patent application is a continuation in part of, and claims priority to, patent application Ser. No. 15/152,193 filed May 11, 2016 and titled “Rapid Diagnostic Kit for Central Nervous System Afflictions.” The subject matter of patent application Ser. No. 15/152,193 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The disclosed subject matter relates generally to the field of medicine and wellness, and, more specifically, to diagnoses for central nervous system afflictions and traumatic brain injuries.

### BACKGROUND

Traumatic brain injury (TBI) is the leading cause of death in the United States in people between the ages of 1 and 44 years and occurs in hundreds of thousands of subjects yearly. Recently, the importance of mild injuries has been recognized as a public health crisis for soldiers in the combat theater, children and young adults in sport activities, and other throughout their normal lives. Symptoms associated with TBI can appear immediately following injury or days or weeks later, and result in wide-ranging physical and psychological deficits including motor impairment, epilepsy, personality change and memory impairment.

Exposure to mild TBI is a common occurrence in athletes on the playing field and soldiers on the battlefield. In fact, playing American Football at higher competitive levels results in documented exposure of 1400 impacts per season for select positions such as linemen and some select positions on both offense and defense with up to 2000 impacts per season. Similarly, mild TBI has been identified as the most common combat-related injury in soldiers returning from present day conflicts in Iraq and Afghanistan and has been referred to as “the signature injury of war”. The lack of a diagnostic test for mild TBI is problematic considering the potential for both enduring cerebral effects (cognitive, neurophysiological, and clinical) and for identification of those at risk for development of (Chronic Traumatic Encephalopathy) CTE later in life.

Traumatic brain injuries (TBI) can occur in several ways and can be characterized by the mechanism of injury, the clinical severity as graded by the Glasgow Coma Scale (GCS), or by the characterization of the structural damage. Generally, injuries are classified as mild, moderate (both of which are considered concussion), or severe depending on the GCS score, which utilizes motor, eye and verbal responses to evaluate the level of cognition. In addition to the GCS score, TBI may be classified according to a clinical injury score based on the level of injury incurred to various key body regions, according to the level of structural damage incurred and according to the patient prognosis based on various prognostic models. Mild TBI is the most common sub category of TBI, with estimates ranging from 1.6 million to 3.8 million annually among athletes in the United States alone. Despite its designation, a mild TBI should not be viewed as an inconsequential injury, as some mild TBI can result in prolonged cognitive, emotional and functional disabilities, significantly impacting the quality of life.

TBI is heterozygous in nature, and no single classification is sufficient in characterizing injury for the purpose of diagnosis and prognosis. Based on the severity of the initial insult, different imaging techniques are used to obtain the necessary information for patient care. In cases of moderate to severe TBI, it is diagnosed through structural abnormalities using techniques such as computed tomography (CT) or conventional magnetic imaging (MRI) which is not very effective to diagnose these subtler irregularities. Mild TBI, such as concussion results in biochemical, metabolic and cellular changes that may be responsible for some long-term problems seen in patients who develop post-concussion syndrome (PCS).

Of all severities of Traumatic Brain Injury (TBI) an estimated 75-85% are categorized as mild TBI, which includes concussion as well as sub-concussion and some blast injuries associated with improvised explosive devices (IEDs). Concussion occurs in a wide variety of activities including sports, such as boxing, American football, rugby, soccer, cheerleading, ice hockey and wrestling, military service; and in association with other exposures such as poorly controlled epilepsy and physical abuse. There is typically full neurologic recovery after a mild TBI; however, 15-30% of subjects develop prolonged neurocognitive and behavioral changes. Concussions are cumulative, and it is believed that the accumulation is one mechanism that can cause dementia.

It is estimated that 1.6-3.8 million concussions occur annually in the United States. Concussion is particularly frequent in American football, where 4.5% of high school, 6.3% of collegiate and 6.6% of professional football players are diagnosed with at least one concussion per season. In addition, the U.S. Department of Defense have diagnosed 339,462 concussions for U.S. Service members since 2000. The true frequency of concussion is probably much greater since concussions are typically unrecognized and sub-clinical, under-reported and can resolve spontaneously without medical care.

There is still no universal consensus regarding the definition of concussion. The 2013 Zurich Consensus Statement on Concussion in Sport proposed that concussion and mild TBI should be viewed as distinct entities. The group defined concussion as a “complex pathophysiological process affecting the brain” and allowed for the presence of neuropathological damage. However, concussive symptoms were largely thought to reflect a functional disturbance, typically resolving spontaneously with no imaging abnormality. In contrast, recent American Academy of Neurology guidelines for sport concussion in 2013 do not separate concussion from mild TBI, defining concussion as a “clinical syndrome of biomechanically induced alteration of brain function, typically affecting memory and orientation, which may involve loss of consciousness”. They noted, however, a lack of consensus in the use of the term, with an overlap in the use of concussion and mild TBI.

Therefore, concussion is currently used in two main ways: (1) to describe a distinct pathophysiological entity with its own diagnostic and management implications, mainly seen in the context of sporting injuries; and (2) to describe a constellation of symptoms that arise after different types of TBI.

There are also problems in retaining concussion as a diagnostic label for the constellation of symptoms that are commonly experienced after TBI. Concussion usually implies a “benign” set of problems that will eventually resolve spontaneously. However, the assumed transience of “concussion” symptoms is problematic, as many patients do not recover quickly and it is difficult to predict long-term outcome after TBI. Even apparently trivial injuries can sometimes have long-term effects with patients reporting similar post concussive symptoms after TBI of all severities.

Standard investigations also do not particularly help in defining “concussion”. Many patients with mild TBI will not undergo neuroimaging and are perhaps wrongly reassured about the concussive nature of their problems without any detailed investigation. Even when there is available neuroimaging, it is easy to be falsely reassured by negative neuroimaging findings. Standard neuroimaging will identify large focal contusions or hemorrhage but normal CT and MRI do not exclude diffuse axonal and vascular injury, both major drivers of poor clinical outcome after TBI. Standard neuroimaging sequences can miss these problems, although more advanced techniques such as susceptibility weighted and diffusion MRI are more sensitive and may identify them.

Blast-related TBI and concussion is among the most frequent injuries sustained by soldiers and other personnel who have served in recent wars in Iraq and Afghanistan. Difficulties of returning personnel with reintegrating into civilian society have been in part been attributed to brain injury that was caused by blast concussions. Reports of blast-related TBI in military personnel deployed to Iraq (Operation Iraqi Freedom) and Afghanistan (Operation Enduring Freedom) has been as high as 19%-23%. Reports of blast-related TBI among military personnel are unprecedented in comparison to military personnel in any other previous war or conflict. Moreover, blast concussion researchers do not enjoy the “unique advantages” that exist in conducting research in sports concussion samples. Like sports concussion samples soldiers and other military personnel represent a group that is at increased risk of sustaining mild TBI. However, blast concussions are only occasionally witnessed, assessments are not typically conducted in a standardized manner and a controlled environment just following the blast, and only rarely are personnel systematically followed during acute, subacute, and post-acute phases like in sports-related concussion.

Concerns have been noted about the reliability of retrospective self-report of blast events and TBI screening in military personnel. With few exceptions, evaluators of blast concussion are not able to corroborate acute-stage injury characteristics and cognitive performances immediately after blast concussions, which impede their ability to effectively diagnose blast-related concussions. Therefore, the evaluators of blast concussion confront a dilemma that can be solved by this diagnostic: blast concussion has been identified as a common hazard of the recent wars in Iraq and Afghanistan, but the elucidation and frequency associated with historical blast concussions is obscured by the unknown reliability and validity of self-report information obtained through contemporary TBI screening methods.

Mild TBI is usually caused by an impact to the head (contact loading) that induces rotational acceleration of the brain (inertial loading). In some patients, mild TBI occurs without an impact to the head such as after rapid rotational acceleration of the head in restrained occupants during a motor vehicle crash. At a neurophysiological level, these mechanical and inertial forces result in the stretching of white matter axons, leading to diffuse axonal injury.

It has long been understood that mild TBI, common in the sport of boxing can lead to dementia syndrome (dementia pugilistica) that includes Parkinson's disease-like motor signs and cognitive symptoms that include bradyphrenia (slowed thinking), confusion, and memory impairment. It is becoming evident that mild TBI experienced by football players is associated with chronic traumatic encephalopathy (CTE), a mid-life dementing disorder evidenced upon autopsy as prominent diffuse neurofibrillary tangles; a hallmark pathologic brain lesion observed in several other neurodegenerative diseases.

The underlying pathophysiology of mild TBI remains undetermined and as a result there is no efficient diagnostic, prognostic or therapeutic strategies currently available. Studies have begun to investigate mild TBI at the cellular and molecular level, as shortcomings in the current brain imaging techniques and flawed clinical diagnostic approaches have increased the appeal for a biochemical assay to diagnose mild TBI. For example, diagnosis and prognosis in moderate and severe TBI using conventional imaging techniques is informative. Specifically the goal of this approach is to uncover a single marker or panel of markers to aide in early detection and diagnosis, as well as potentially predict patient outcomes.

Consequently, there is an urgent medical need to develop a biochemical diagnostic for concussions. Currently, the only available TBI or concussion tests are either cognitive, which can be falsely interpreted, or require testing equipment that can only be found in hospitals or in testing laboratories. There is currently no non-invasive “field” concussion diagnostic that can be deployed on a sports field, in a school, workplace or battlefield. Therefore, a need exists to overcome the problems with the prior art as discussed above, and particularly for a more efficient and expeditious way of diagnosing concussions.

Another well-known affliction of the central nervous system is Chronic Traumatic Encephalopathy (CTE), which is a progressive neurodegenerative disease currently only diagnosed at autopsy and subsequent neuropathological examination. Since this condition can only be effectively diagnosed after autopsy, there is an urgent clinical need to establish a diagnostic tool that can effectively diagnose CTE while the patient is alive, thereby permitting or developing an effective treatment regimen.

CTE has been diagnosed in a wide range of individuals with a history of head trauma, although the number and severity of impacts is often unclear, ranging from athletes playing American football, soccer, hockey, boxers and wrestlers to soldiers who have received battlefield injuries. Due to the variety of individuals afflicted by CTE, new evidence suggests that CTE is more common than previously thought. Based on some studies, a conservative estimate of lifetime prevalence of CTE in American football players is at least 3.7%. Estimates of CTE prevalence in retired boxers is 20%. Considering the number of individuals actively engaged in contact sports such as football or exposure to explosive devices on the battlefield, it is clear that CTE represents a clear public health risk. There is mounting evidence demonstrating that concussions or mild traumatic brain injury have more serious complications, particularly when they occur repeatedly. Sports-related CTE manifests as a progressive worsening of cerebral neurological symptoms, initiated by repetitive concussions and sub-concussions. While the exact incidence and prevalence remains unknown, recent epidemiological data suggests that 17% of individuals subjected to repeated concussive events may develop CTE; however, the severity and the frequency of repetitive injury needed to cause CTE remains elusive.

Patients with CTE typically show signs of dizziness, unsteady gait, fatigue, and dysarthria. In addition, they may also demonstrate cognitive and psychological symptoms, such as memory loss, attention loss, difficulty in concentration, slow information processing, confusion, loss of judgement, irritability, emotional distress, a progressive slowing of movement, tremor, deafness, dysphagia, ptosis, and other ocular abnormalities.

Clinical deterioration in CTE typically occurs in three stages: (1) Affective disturbances and psychotic symptoms, (2) Social instability, erratic behavior, memory loss, and initial signs of Parkinson's disease, (3) General cognitive dysfunction, the progression of dementia, speech, gait abnormalities, or full-blown Parkinson's disease. The diagnosis of CTE is based on a history of repetitive concussions, which may have been subclinical, evidence of disease progression, and a neurological exam. CT and MRI scans can be helpful for excluding other neurological conditions such as chronic subdural hematoma or brain tumor.

Consequently, a need also exists to overcome the problems with the prior art as discussed above, and particularly for a more efficient and expeditious way of diagnosing CTE in patients.

Furthermore, many neurodegenerative disorders share a common pathophysiological pathway involving axonal degeneration despite different etiological triggers. Multiple Sclerosis (MS) is the most common autoimmune disease of the central nervous system in young adults affecting about 30 in 100,000. The majority of MS-patients face the relapsing remitting form of the disease, in which the attacks are usually a sign of acute exacerbation of the inflammation. After an average of 19.1-21.4 years, about one third of the patient's progress to the secondary phase of the disease, which is characterized by slowly accumulating disability with or without acute exacerbations. However, about 11%-18% of the patients have primary progressive multiple sclerosis (PPMS) with continuous slowly accumulating disability.

The pathological hallmark of MS is inflammation induced demyelination and subsequent axonal loss, which may be initially accompanied by re-myelination in part of the lesions. Pathological studies revealed different types of plaques depending on the stage of the inflammatory reaction (active plaques, slowly expanding lesions, inactive plaques and re-myelinated shadow plaques). Histopathological examination of acute MS lesions revealed different patterns of tissue injury indicating possible different mechanisms of the disease cause. T cell infiltrates and macrophage-associated tissue injury (pattern 1); antibody and complement-mediated immune reactions against cells of the oligodendrocyte lineage and myelin (pattern 2); hypoxia-like injury, resulting either from inflammation-induced vascular damage or macrophage toxins that impair mitochondrial function (pattern 3); and a genetic defect or polymorphism resulting in primary susceptibility of the oligodendrocytes to immune injury (pattern 4).

Amyotrophic lateral sclerosis (ALS) is a progressive neurodegenerative disease that affects nerve cells in the brain and the spinal cord. Since the first report of ALS more than 100 years ago, the main pathophysiological mechanisms still remain unclear. Degeneration of the motor neurons is followed by an inflammatory reaction with gliosis and accumulation of activated microglia and astrocytes with the production of cytotoxic molecules and inflammatory cytokines like TNF-α and IL-1β. Glial cells also play an important role in the pathophysiology of ALS. Due to a deficient astrocyte-specific glutamate transporter (GLT-1) or excitatory amino acid transporter-2 (EAAT2), the astrocytes fail to clear up the glutamate leading to exacerbation of the glutamine excitotoxicity. Moreover, the role of astrocytes and microglia is supported by the observed increase in production of reactive oxygen species (ROS), nitric oxide and interferon-Υ. The role of microglia is more evident in the late stages on the disease.

Bacterial meningitis is a devastating infection associated with high mortality and morbidity particularly in the neonatal population. Prompt diagnosis and treatment are essential to achieving good outcomes in affected individuals. While the overall incidence and mortality have declined over the last several decades, morbidity associated with neonatal meningitis remains virtually unchanged.

Meningitis is a syndrome classically characterized by some combination of neck stiffness, headache, fever and altered mental status; other symptoms including nausea, vomiting and photophobia are frequently observed as well. Mortality may vary widely according to cause and setting with rates of 3-30% for bacterial meningitis depending on the organism. Aseptic meningitis (usually referring to viral meningitis but also encompassing other “culture-negative” types of meningitis) is generally considered a benign, self-limited disease with low mortality.

CT and MRI may be considered as adjunctive diagnostic tests but are generally nonspecific and show meningeal enhancement. Imaging may be helpful in cases of focal neurologic deficits, particularly when a tuberculoma or cryptococcoma is suspected. Standard diagnostic testing of cerebro-spinal fluid (CSF) includes: white blood cell count with differential, total protein, and CSF/blood glucose, used in conjunction with patient history and epidemiology to support potential diagnosis. Total protein and white blood cell (WBC) counts reflect inflammation in the CSF while decreased glucose CSF/blood ratio is a sign of glucose consumption by an active infection. These common laboratory tests cannot be the lone laboratory method of diagnosis and while overlap in their values among different diagnoses does occur, general trends emerge and are useful as they help the clinician to focus on particular possible diagnoses. Importantly, up to 40% of persons with Cryptococcus may have an unremarkable CSF WBC count which can mistakenly delay the diagnosis.

Patients with meningitis are often difficult to classify into bacterial or benign viral meningitis. On admission, patients with bacterial meningitis do not always display the typical clinical signs and laboratory findings can be confounding. CSF leukocyte count in bacterial meningitis can be lower than 100/ul indicating a sever course. CSF leukocyte count differentiation is also imprecise. More than 30% of the bacterial meningitis patients with CSF leukocyte counts less than 1,000/ul display a CSF lymphocytosis instead of the typical granulocytosis.

Facing these difficulties, a bacterial meningitis score was designed. While this score allowed the identification of all but two of more than 121 pediatric bacterial meningitis patients, too many viral meningitis patients showed scores that were indicative of bacterial meningitis. Similarly, Gram stain, the occurrence of seizure at or before presentation, peripheral leukocyte count, CSF leukocytes and protein CSF concentration could not correctly classify all pediatric bacterial meningitis patients. Of those who were, according to this score, at risk for bacterial meningitis, more than 40% and to be reclassified as viral meningitis. A score built of C-reactive protein and protein CSF content falsely classified as many as 16 of 71 pediatric patients with viral meningitis.

Consequently, a further need exists to overcome the problems with the prior art as discussed above, and particularly for a more efficient and expeditious way of diagnosing MS, ALS and meningitis in patients.

### SUMMARY

This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

A method and system for diagnosing concussions, TBI, CTE, ALS, MS, meningitis and other CNS afflictions is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a diagnostic kit includes a tube containing a predefined amount of lyophilized biomarker-specific antibody conjugated to a conjugate with color functionality, a fluid for mixing with the lyophilized biomarker-specific antibody conjugated to the conjugate within the tube, so as to reconstitute the lyophilized biomarker-specific antibody conjugated to the conjugate, resulting in a reconstituted mixture, and a swab comprising an absorbent hydrophobic material, the swab configured for absorbing cerebrospinal fluid from a patient, wherein when the swab that has absorbed cerebrospinal fluid is placed in the tube containing the reconstituted mixture, the swab tip is configured to change color. In an additional embodiment, a method for diagnosing concussions, TBI, CTE, ALS, MS, meningitis and other CNS afflictions is also provided.

To the accomplishment of the above and related objects, the claimed subject matter may be embodied in the form illustrated in the accompanying drawings, attention being called to the fact, however, that the drawings are illustrative only, and that changes may be made in the specific construction illustrated and described within the scope of the appended claims. The foregoing and other features and advantages of the claimed subject matter will be apparent from the following more particular description of the preferred embodiments, as illustrated in the accompanying drawings.

### BRIEF DESCRIPTION OF THE DRAWINGS

The accompanying drawings, which are incorporated in and constitute part of this specification, illustrate embodiments of the claimed subject matter and together with the description, serve to explain the principles of the disclosed embodiments. The embodiments illustrated herein are presently preferred, it being understood, however, that the claimed subject matter is not limited to the precise arrangements and instrumentalities shown, wherein:

FIG. 1 is a flowchart showing the control flow of the process 100 for diagnosing concussions, TBI, CTE, ALS, MS, meningitis and other CNS afflictions, according to one embodiment.

FIGS. 2, 3, and 4 are illustrations of various swabs useful for implementing the system for diagnosing concussions, TBI, CTE, ALS, MS, meningitis and other CNS afflictions, according to one embodiment.

FIGS. 5 and 6 are illustrations of a swab used in the process for diagnosing concussions, TBI, CTE, ALS, MS, meningitis and other CNS afflictions, according to one embodiment.

FIGS. 7 and 8 are illustrations of a tube used in the process for diagnosing concussions, TBI, CTE, ALS, MS, meningitis and other CNS afflictions, according to one embodiment.

FIGS. 9, 10 11, and 12 are illustrations of a swab in various stages of the process for diagnosing concussions, TBI, CTE, ALS, MS, meningitis and other CNS afflictions, according to one embodiment.

FIG. 13 is a flowchart showing the control flow of an alternative process 1300 for diagnosing concussions, TBI, CTE, ALS, MS, meningitis and other CNS afflictions, according to one embodiment.

FIGS. 14, 15, 16, 17, 18, and 19 are illustrations of a first embodiment of a nitrocellulose membrane in various stages of the process for diagnosing concussions, TBI, CTE, ALS, MS, meningitis and other CNS afflictions, according to one embodiment.

FIGS. 20, 21, 22, 23 are illustrations of a second embodiment of a nitrocellulose membrane in various stages of the process for diagnosing concussions, TBI, CTE, ALS, MS, meningitis and other CNS afflictions, according to one embodiment.

FIG. 24 is an illustration of a third embodiment of a nitrocellulose membrane for diagnosing concussions, TBI, CTE, ALS, MS, meningitis and other CNS afflictions, according to one embodiment.

FIGS. 25, 26, 27 are illustrations of a fourth embodiment of a nitrocellulose membrane in various stages of the process for diagnosing concussions, TBI, CTE, ALS, MS, meningitis and other CNS afflictions, according to one embodiment.

### DETAILED DESCRIPTION

The disclosed embodiments are directed to a rapid, inexpensive and easy-to-use diagnostic test for concussions, TBI, CTE, ALS, MS, meningitis and a variety of central nervous system afflictions, among other things. The disclosed embodiments improve over the prior art by providing a diagnostic kit that cannot be falsely interpreted, does not require testing equipment, does not require a trip to a hospital or testing laboratory, is non-invasive and can be easily deployed at a sports field, school, workplace or battlefield. The disclosed embodiments further improve over the prior art by providing a diagnostic kit that provides a definitive result regarding concussions, TBI, CTE, ALS, MS, meningitis and a variety of central nervous system afflictions, regardless of the lack of universal consensus regarding the definition of a concussion, TBI, and other central nervous system afflictions.

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering, or adding additional stages or components to the disclosed methods and devices. Accordingly, the following detailed description does not limit the disclosed embodiments. Instead, the proper scope of the disclosed embodiments is defined by the appended claims.

Cerebrospinal Fluid Biomarkers

The methods disclosed herein utilize the rapid detection of specific biomarkers which are released in high concentrations into the Cerebrospinal Fluid (CSF) surrounding the brain upon brain injury, including concussion. The insult causes micro-tears in the Brain-Blood Barrier (BBB) causing CSF to leak from the brain into the nasal or auditory passages. This fluid can be collected and utilized as the test material for the concussion diagnostic and even milder concussive events.

CSF biomarkers may be exploited in the disclosed diagnostic kit to assess TBI, the severity of TBI and the potential outcome of the patient. Various biomarkers can be detectable in the CSF resulting from the damage of the BBB following TBI. One of these biomarkers is the protein S100B.

S100B is traditionally referred to as an astrocytic protein, and has been demonstrated as a potential biomarker of TBI. The S100 proteins are a family of Ca⁺²-binding proteins that help regulate intracellular levels of Calcium. The first S100 proteins were originally discovered in 1965 and two homodimeric proteins S100-A1 (consisting of 2α subunits) and S100-B1 (consisting of 2β subunits) were subsequently identified. One study demonstrated that amateur boxers who suffered from minor head injuries found S100B levels were elevated in the CSF as assayed via lumbar puncture. The magnitude of S100B elevation has been correlated to the severity of TBI, scoring on the Glasgow Coma Scale, as well as radiological findings at hospital admission. This protein can be used in the disclosed diagnostic process as a target biomarker for detection and possible elucidation of severity of TBI, including concussion and mild concussion.

A second biomarker that can be exploited in the disclosed diagnostic process for the detection of concussion and TBI is Υ-enolase (Neuron Specific Enolase [NSE]). In studies of TBI, NSE was found to be elevated in CSF, with a higher magnitude of elevation corresponding to higher mortality and a more severe score based on the Glasgow Coma Scale for both adults and children.

Another TBI biomarker to be used with the disclosed diagnostic process for the detection of TBI is UCH-L1 (ubiquitin C-terminal hydrolase), also known as neuronal specific gene product 9.5 and its breakdown product is a novel biomarker naturally expressed in neurons and has been found to be elevated in CSF in response to TBI. The elevation of these breakdown products have been correlated to the severity and provides more predictive power for the IMPACT outcome calculator for patients with severe TBI. Recent studies have demonstrated significant increases in CSF levels of UCH-L1 after controlled impact TBI in rats. A pilot study of UCH-L1 in mild to moderate TBI, UCH-L1 was shown to be elevated 1-hour post-injury and was found to be correlated with injury severity, the GCS score and positive lesions on CT imaging.

Non-erythroid αII-spectrin is a well-known component of the cytoskeleton of all non-erythroid tissues. Neurons contain the highest concentrations in the subaxolemmal compartment and presynaptic terminals. After TBI, this protein is protealytically cleaved from the intact brain spectrin. The resulting 145-kDa fragment is specific calpain and the 120-kDa fragment is associated with caspase-3-mediated cleavage, both of which can serve as biomarkers for TBI and can be used as target biomarkers in the disclosed diagnostic process.

Amyloid β is a peptide that normally exists in monomeric form. Following injury, it polymerizes into plaques that are toxic to the tissue nearby. Rapid induction of the marker is seen in the brain within the first day post-injury in experimental animals and remains detectable through day 14 post-injury. This protein is currently used as a histological marker of axonal injury, however since it exists in high concentrations in the CSF it would make an ideal target biomarker for the disclosed diagnostic process.

Glial Fibrillary Acidic Protein (GFAP) is an acidic filament protein located within astrocytes and otherwise not found outside the Central Nervous System. Following experimental models of TBI in rats, increased GFAP mRNA expression bad been observed for at least 11 days following the initial insult. Experimental subjects with unfavorable outcomes demonstrated higher concentration GFAP than favorable subject outcomes at 11 and 14 days. Due to the higher concentration levels and the lack of GFAP in any other compartment aside from the nervous system, this protein serves as an ideal biomarker for detection in CSF using the disclosed diagnostic process for the determination of concussion and TBI.

Studies performed in both animals and humans have demonstrated that the amyloid precursor protein (APP) accumulates in neurons and axons after brain trauma and can cause axonal damage. In several studies for TBI, accumulation of APP occurs within hours of the initial insult and also occurs in patients with mild TBI. CSF levels of APP increase substantially in the first week following TBI. Due to the accumulation of this protein and its preferential expression in nervous tissue, this can be a potential biomarker for detection in a CSF lateral flow or vertical flow concussion diagnostic test.

The diagnostic process and kit described herein for concussions, TBI, CTE and other central nervous system afflictions is designed to be used with any of the biomarkers described herein, or any combination of these biomarkers. In addition, the antibodies that recognize and bind to these specific biomarkers can be conjugated to any of the conjugates listed herein or any other conjugate as well as any combination of the listed conjugates or any other conjugate.

The Tau Protein

The method and system for diagnosing a concussion (as well as other CNS afflictions) without invasive techniques or complicated wet-laboratory equipment is disclosed herein. The methods utilize the rapid detection of the tau protein which is released in high concentrations into the Cerebrospinal Fluid (CSF) surrounding the brain upon brain injury, including concussion. The insult causes micro-tears in the Brain-Blood Barrier (BBB) causing CSF to leak from the brain into the nasal or auditory passages. This fluid can be collected and utilized as the test material for the concussion diagnostic and even milder concussive events. In addition, this diagnostic kit can be used to diagnose bacterial meningitis, Amyotrophic Lateral Sclerosis (ALS) and clinically active multiple sclerosis, all of which have demonstrated to have elevated tau protein levels in the CSF.

Tau is a microtubule-associated protein localized in neuronal cells and functions as a major structural element in the axonal cytoskeleton. Total tau is abundant in the central nervous system. The hyperphosphorylation of tau is associated with several neurodegenerative diseases that are referred to as tauopathies. Tau levels also markedly influence the pathophysiology of TBI and can serve as an informative biomarker for TBI, including concussion.

After TBI, tau is proteolytically cleaved and gains access to the CSF. In one study CSF levels of c-tau were significantly elevated in TBI patients compared with control patients, and these levels correlated with clinical outcomes. Several studies have consistently demonstrated that tau CSF levels, which have been closely linked with the presence of axonal injury, increased intracranial pressure, and clinical outcome, are increased in TBI and concussion patients as compared to normal controls. And it has been demonstrated that there is a rapid rise in concentration of tau in CSF following TBI. A common occurrence associated with concussion or TBI is CSF rhinorrhea or the leakage of CSF from the nasal cavity following an intracranial insult. Normally CSF is confined to the space around the brain and spinal cord. Due to its proximity to the sinus and nasal cavity any damage to the Brain-Blood Barrier (BBB), which occurs in concussion will cause the fluid and tau protein to leak and drain out through the nose. Sometimes, instead of the nose, it can leak through the ears where it is known as CSF otorrhea.

CSF has several important functions, such as acting as a shock absorber and thereby protecting the brain and spinal cord during impact, keeping the brain afloat within the cranial cavity, and draining away large proteins and other substances that are not carried out by the vasculature. This most obvious symptom is quite frequently missed by the patient as mistakenly believed to be nasal mucus (runny nose).

In addition, the BBB hinders the assessment of biochemical changes in the brain by use of biomarkers in the blood making the CSF a much more ideal target. However, the BBB integrity can become compromised which can result in an increase in the levels of brain specific proteins in the blood. Some of these biomarkers also get proteolytically degradation in the blood and their levels might be affected by clearance from the blood via the liver or kidney. Nevertheless, the literature on potential peripheral blood biomarkers of brain injury patients with TBI is abundant. Even with these limitations, serum tau levels have recently been demonstrated to rapidly increase following mild TBI and declined after 6 hours post insult. The tau protein levels were also severity-dependent at 1 and 6 hours after TBI. These levels were higher in the severe TBI group than in the mild TBI at 1 and 6 hours.

CTE Biomarkers

The best established CSF biomarker for tangle pathology is phosphorylated tau protein. On the microscopic level, neurofibrillary tangles—the aggregation of the phosphorylated protein tau, is one of the hallmarks of CTE. The inclusions form in both neurons and glial cells. The neurofibrillary tangles in CTE are irregularly distributed with a tendency to form around blood vessels. When the neurofibrillary tangles are diffusely distributed, they are preferentially found in the superficial layers of the cerebral cortex.

It has been demonstrated that the hyperphosphorylated variant of tau impairs its binding to microtubules and its capacity to promote microtubule assembly, resulting in its self-aggregation into neurofibrillary tangles, microtubule disorganization, and impaired transport along axonal microtubules. The hyperphosphorylation is thought to result from an imbalance in the function of several protein kinases and phosphatases.

This phosphorylated/hyperphosphorylated variant of the tau protein may serve as a lateral flow or vertical flow biomarker as a biochemical indicator of CTE in high risk patients, i.e. patients that have been subjected to repeated mild TBI events. In order to test phosphorylated/hyperphosphorylated tau as a biomarker for CTE, a specific antibody that recognizes the phosphorylated variant of tau will be employed. The antibody will specifically recognize Ser202/Thr205 phosphorylated variant of tau as this variant is the most commonly associated with neurofibrillary tangles and tauopathies.

In addition to phosphorylated/hyperphosphorylated tau protein, a DNA-binding protein, TDP-43 forms concentration-dependent inclusions in CTE-affected brains and spinal cords. TDP-43 accumulation can be widespread and is found in several grey matter structures, such as the brainstem, basal ganglia, and cortical areas, as well as in subcortical white matter. While the exact function of TDP-43 is unknown, its overexpression causes neuronal degeneration and cell death in animal models. One hypothesis is that mild TBI causes axonal sheer with cytoskeleton disruption. As part of the injury response, TDP-43 is upregulated and binds to neurofilament mRNA in an attempt to stabilize the transcript. Since this protein is prone to aggregation, pathological TDP-43 deposits form, resulting in cell death. The increase in concentration upon injury and the presence of this protein in the CSF can make TDP-43 a candidate for use in this invention for the indication of CTE.

Other Biomarkers

In addition to the biomarkers listed above, the increase in concentration or the change in normal physiological localization of the listed biomarkers may cause a change in concentration or in the normal physiological localization of another biomarker, thereby leading to the indirect assay of one of the biomarkers listed above. For example, the increased concentration of tau protein in the CSF may lead to the increase in expression or localization of protein X into the CSF, this would be an indirect way to use protein X as a biomarker to assay for tau protein. These downstream “indirect” biomarkers are covered under this patent application.

Cost remains a significant barrier for many new molecular diagnostics both in high-income and low/middle income countries. Excluding reference laboratories, most local hospital microbiology labs are costs to a healthcare system, not a revenue generator. A new relatively expensive assay may cost more for a microbiology laboratory. A second ironic barrier to adoption is the standard Good Clinical Lab Practice of every laboratory internally validating a new assay. For fully automated U.S. FDA-approved molecular assays, this slows adoption for relatively rare diseases where validation takes significant time and effort. Third, as new molecular tests become available, how best to utilize such testing in a cost-effective manner in high- and low-income settings needs to be explored. Based on these limitations, there is an urgent need for a rapid, inexpensive diagnostic tool.

Neurodegenerative Disorders

Many neurodegenerative disorders share a common pathophysiological pathway involving axonal degeneration despite different etiological triggers. Analysis of cytoskeletal markers like tau in CSF is a useful approach to detect the process of axonal damage and its severity during disease course.

Multiple Sclerosis (MS) is the most common autoimmune disease of the central nervous system in young adults affecting about 30 in 100,000. The majority of MS-patients face the relapsing remitting form of the disease, in which the attacks are usually a sign of acute exacerbation of the inflammation. After an average of 19.1-21.4 years, about one third of the patient's progress to the secondary phase of the disease, which is characterized by slowly accumulating disability with or without acute exacerbations. However, about 11%-18% of the patients have primary progressive multiple sclerosis (PPMS) with continuous slowly accumulating disability.

The pathological hallmark of MS is inflammation induced demyelination and subsequent axonal loss, which may be initially accompanied by re-myelination in part of the lesions. Pathological studies revealed different types of plaques depending on the stage of the inflammatory reaction (active plaques, slowly expanding lesions, inactive plaques and re-myelinated shadow plaques). Histopathological examination of acute MS lesions revealed different patterns of tissue injury indicating possible different mechanisms of the disease cause. T cell infiltrates and macrophage-associated tissue injury (pattern 1); antibody and complement-mediated immune reactions against cells of the oligodendrocyte lineage and myelin (pattern 2); hypoxia-like injury, resulting either from inflammation-induced vascular damage or macrophage toxins that impair mitochondrial function (pattern 3); and a genetic defect or polymorphism resulting in primary susceptibility of the oligodendrocytes to immune injury (pattern 4).

It has been demonstrated that tau protein is highly elevated in CSF in primary multiple sclerosis compared to individuals without MS. Therefore, the disclosed embodiments can be used in the diagnosis of primary MS. CSF can be isolated from the potential MS patient by a routine spinal tap and applied to either tau protein diagnostic kit. Since the tau protein has been elevated to detectable levels in the CSF in MS patients, a positive test result will be a rapid method to indicate high levels of tau in the CSF which will be an indication that the individual is suffering from MS. When used in conjunction with the other battery of MS diagnostics, this embodiment can serve as a very powerful tool to aid in the diagnosis of MS.

Since the first report of ALS more than 100 years ago, the main pathophysiological mechanisms still remain unclear. Degeneration of the motor neurons is followed by an inflammatory reaction with gliosis and accumulation of activated microglia and astrocytes with the production of cytotoxic molecules and inflammatory cytokines like TNF-α and IL-1β. Glial cells also play an important role in the pathophysiology of ALS. Due to a deficient astrocyte-specific glutamate transporter (GLT-1) or excitatory amino acid transporter-2 (EAAT2), the astrocytes fail to clear up the glutamate leading to exacerbation of the glutamine excitotoxicity. Moreover, the role of astrocytes and microglia is supported by the observed increase in production of reactive oxygen species (ROS), nitric oxide and interferon-Υ. The role of microglia is more evident in the late stages on the disease.

As far as the disclosed embodiment's functionality in diagnosing ALS, some studies have demonstrated higher levels of tau compared to non-ALS patients. Studies have also demonstrated that patients in the earlier disease stages exhibit a higher level of tau than those with the advanced disease. Since the tau protein has been elevated to detectable levels in the CSF in ALS patients, a positive test result will be a rapid method to indicate high levels of tau in the CSF which will be an indication that the individual is suffering from ALS. When used in conjunction with the other battery of ALS diagnostics, this embodiment can serve as a very powerful tool to aid in the diagnosis of ALS.

Bacterial meningitis is a devastating infection associated with high mortality and morbidity particularly in the neonatal population. Prompt diagnosis and treatment are essential to achieving good outcomes in affected individuals. While the overall incidence and mortality have declined over the last several decades, morbidity associated with neonatal meningitis remains virtually unchanged.

Meningitis is a syndrome classically characterized by some combination of neck stiffness, headache, fever and altered mental status; other symptoms including nausea, vomiting and photophobia are frequently observed as well. Mortality may vary widely according to cause and setting with rates of 3-30% for bacterial meningitis depending on the organism. Aseptic meningitis (usually referring to viral meningitis but also encompassing other “culture-negative” types of meningitis) is generally considered a benign, self-limited disease with low mortality.

CT and MRI may be considered as adjunctive diagnostic tests but are generally nonspecific and show meningeal enhancement. Imaging may be helpful in cases of focal neurologic deficits, particularly when a tuberculoma or cryptococcoma is suspected. Standard diagnostic testing of CSF includes: white blood cell count with differential, total protein, and CSF/blood glucose, used in conjunction with patient history and epidemiology to support potential diagnosis. Total protein and WBC counts reflect inflammation in the CSF while decreased glucose CSF/blood ratio is a sign of glucose consumption by an active infection. These common laboratory tests cannot be the lone laboratory method of diagnosis and while overlap in their values among different diagnoses does occur, general trends emerge and are useful as they help the clinician to focus on particular possible diagnoses. Importantly, up to 40% of persons with Cryptococcus may have an unremarkable CSF WBC count which can mistakenly delay the diagnosis.

Cost remains a significant barrier for many new molecular diagnostics both in high-income and low/middle income countries. Excluding reference laboratories, most local hospital microbiology labs are costs to a healthcare system, not a revenue generator. A new relatively expensive assay may cost more for a microbiology laboratory. A second ironic barrier to adoption is the standard Good Clinical Lab Practice of every laboratory internally validating a new assay. For fully automated U.S. FDA-approved molecular assays, this slows adoption for relatively rare diseases where validation takes significant time and effort. Third, as new molecular tests become available, how best to utilize such testing in a cost-effective manner in high- and low-income settings needs to be explored. Based on these limitations, there is an urgent need for a rapid, inexpensive diagnostic tool for meningitis.

Patients with meningitis are often difficult to classify into bacterial or benign viral meningitis. On admission, patients with bacterial meningitis do not always display the typical clinical signs and laboratory findings can be confounding. CSF leukocyte count in bacterial meningitis can be lower than 100/ul indicating a sever course. CSF leukocyte count differentiation is also imprecise. More than 30% of the bacterial meningitis patients with CSF leukocyte counts less than 1,000/ul display a CSF lymphocytosis instead of the typical granulocytosis.

Facing these difficulties, a bacterial meningitis score was designed. While this score allowed the identification of all but two of more than 121 pediatric bacterial meningitis patients, too many viral meningitis patients showed scores that were indicative of bacterial meningitis. Similarly, Gram stain, the occurrence of seizure at or before presentation, peripheral leukocyte count, CSF leukocytes and protein CSF concentration could not correctly classify all pediatric bacterial meningitis patients. Of those who were, according to this score, at risk for bacterial meningitis, more than 40% and to be reclassified as viral meningitis. A score built of C-reactive protein and protein CSF content falsely classified as many as 16 of 71 pediatric patients with viral meningitis.

CSF leakage renders one more susceptible to infections of the brain such as subdural or epidural infections due to Neiseeria meningitis, Staphylococcus pneumonia or Staphylococcus aureus. On the other hand, meningitis infections can compromise the dura resulting in CSF leakage. Additionally, CNS neoplasms and or abscesses within the brain can also result in a compromised dura and blood brain barrier and may cause leakage of CSF. Therefore, the disclosed embodiments could be used to collect the CSF fluid that has been leaked though the BBB and be used to detect the tau protein thereby acting as a preliminary indicator for the above said conditions.

Tau protein has been documented to be elevated in both bacterial meningitis and encephalitis, making tau protein an ideal target for a biomarker for bacterial meningitis. In addition, this makes the disclosed embodiments an ideal candidate as a diagnostic tool for bacterial meningitis. Since a rapid, accurate diagnosis of bacterial meningitis is paramount to treatment and the survivability of bacterial meningitis, the rapid diagnosis using the disclosed embodiments will help differentiate bacterial from viral meningitis leading to a better outcome for the individual suffering from the more lethal form, bacterial meningitis.

CSF can be isolated from the afflicted individual and applied to the disclosed embodiments. If the tau protein is detected in the CSF, this would be a clear indication that the individual is suffering from bacterial meningitis where CSF tau protein levels are elevated to detectable levels compared to low levels of tau in viral meningitis. Any of the disclosed diagnostic kit designs can be used, and ideally the CSF should be used by a routine spinal tap as the source material for the diagnostic kit. Taken together with the other symptoms that the patient displays, medical history and other battery of bacterial meningitis diagnostics, this embodiment can serve as a powerful tool to assist in the diagnosis of bacterial meningitis.

The Diagnostic Kit

The method and system for diagnosing concussions, TBI, ALS, MS, and meningitis (as well as other CNS afflictions) without invasive techniques or complicated wet-laboratory equipment comprises a diagnostic kit that includes a color-changing swab 502 and a tube 702. In a first embodiment, the diagnostic kit detects a specific biomarker or combination of biomarkers (such as S100B, NSE, UCH-L1, Non-erythroid αII-spectrin, Amyloid β, GFAP, and APP) rapidly by enabling the swab to change color, indicating the presence of CSF. In the case of a rapid diagnostic tool for CTE, the biomarker panel consists of phosphorylated/hyperphpsphorylated Tau protein and/or TDP-43 protein.

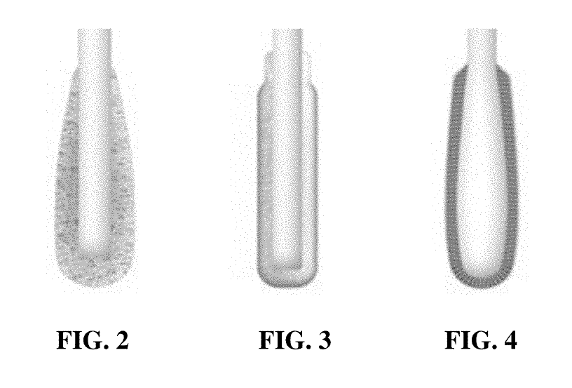

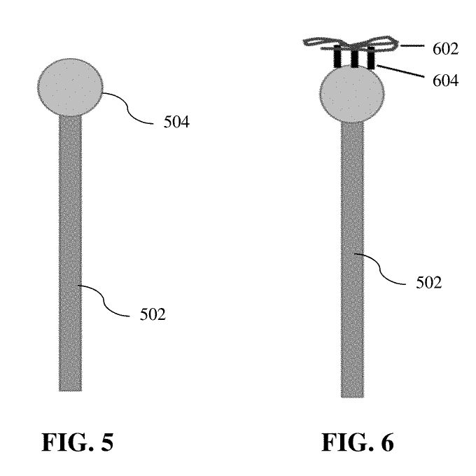

The method may employ a polyurethane, foam-tipped hydrophobic nasopharyngeal swab (see FIG. 3) for nasal collection to collect CSF that has been discharged from the nose or from the ears following insult. The CSF may also be collected from a spinal tap, peripheral blood, etc. A collar may be added at 5.5 cm as a guide for maximum insertion depth. For ear collection, a flocked hydrophobic ear swab (see FIG. 4) or a fiber-tipped swab (see FIG. 2) may be employed, inserted gently into the ear and rotated to collect the discharged CSF. The swab may be hydrophobic foam so that the tau protein remains bound.

The swab containing the CSF will have the target biomarker(s), which, after insult, is typically found in elevated concentrations in the CSF and is not found in the nasal passage nor the ear canal. Tau is specifically housed in neuronal tissue and CSF. The only way that tau will be detected will be due to increased concentrations of tau due to intracranial insult and due to leakage of CSF caused by intracranial insult. The method employs buffers where the salt concentration in the tube 702 is below 750 mM (milli-molars) which will not force any secondary structure formation of the tau protein. The buffers will also be devoid of any polyanions which can induce the aggregation of tau through the masking of charged amino acids in the tau protein potentially leading to the masking of the epitope and failure of the antibody binding. The method also employs an antibody or antibodies that are designed to bind irreversibly to the target concussion biomarker. The antibody/antibodies will have a colorimetric color change indicator that will change color based on the specific biomarker.

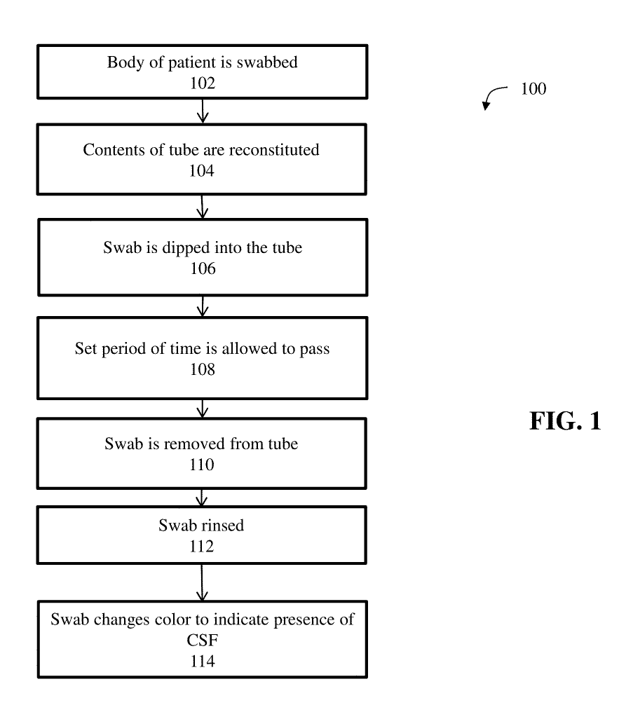

FIG. 1 is a flowchart showing the control flow of the process 100 for diagnosing TBI, concussions, MS, ALS and meningitis, as well as other CNS afflictions, according to one embodiment. This detection method exploits a unique biochemical feature of the aforementioned biomarkers, which include the tau protein, the S100B protein, NSE, UCH-L1, non-Erythroid αII-spectrin, Amyloid β, GFAP, APP, Ser202/Thr205, Ser202/Thr205 phosphorylated Tau, TDP-43, etc.

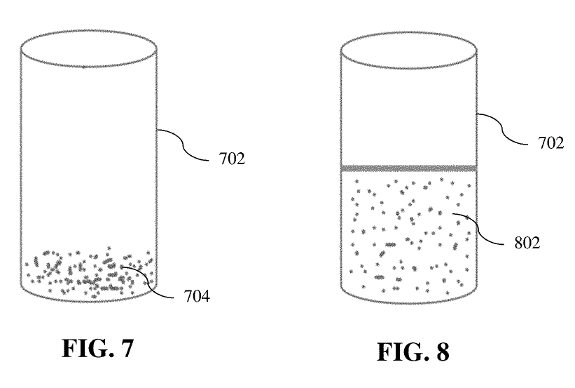

The process of FIG. 1 begins with a step 102 wherein a portion of the body of the patient (ear canal, nasal cavity, etc.) is swabbed with the swab 502. The swab will have a hydrophobic tip 504. The hydrophobic swab tip will bind to the hydrophobic amino acids exposed on the biomarker protein(s) 602 in the CSF, resulting in a very tight and stable hydrophobic-hydrophobic interaction or binding 604 between the swab 504 and said protein(s) 602 or biomarkers (see FIG. 6). The tube 702 of the kit will contain a lyophilized biomarker-specific antibody 704 conjugated to any one or more of the following conjugates: 400 nm red latex nanoparticles, other nanoparticles (including in any of the following colors: ultra violet, plum purple, glacial blue, yellow green, surf green, dragon green, envy green, suncoast yellow, flash red, blue, nile red, orange, red-orange, ded, crimson, dark red, infrared, white, yellow x, pink XC, red Z, pink Y, fluoro-max Eu, sky blue, blue/violet, bright blue, black and yellow-orange), colloidal gold, colloidal silver, quantum dots, fluorescent latex particles (including Alexa Fluor 350, 405, 430, 488, 514, 532, 546, 555, 668, 594, 610, and 647, DyLight 350, 405, 488, 550, 594, 633, 650, 680, 755, and 800, TF1, 2, 3, 4, 5, 6, 7, and 8), up-converting phosphors, enzymes, colloidal carbon and platinum, lipsome-based probes, magnetic particles, or Raman-active tags (see FIG. 7). Alternatively, beads of about 2.8 μm may be used. Most of the aforementioned conjugates have color functionality, which means that the conjugates exhibit a color that is visually detectable with the human eye when present in a minimum amount. As such, the conjugate will serve as the color indicator for a positive test result. The tube 702 of the kit may contain 0.5 mg of the lyophilized biomarker-specific antibody 704. In this document, the term conjugated refers to linking, bonding, coupling, covalently coupling or a compound formed by the joining of two or more chemical compounds.

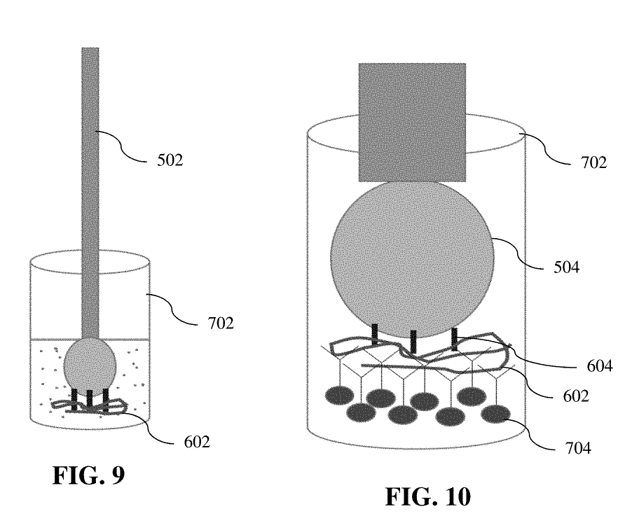

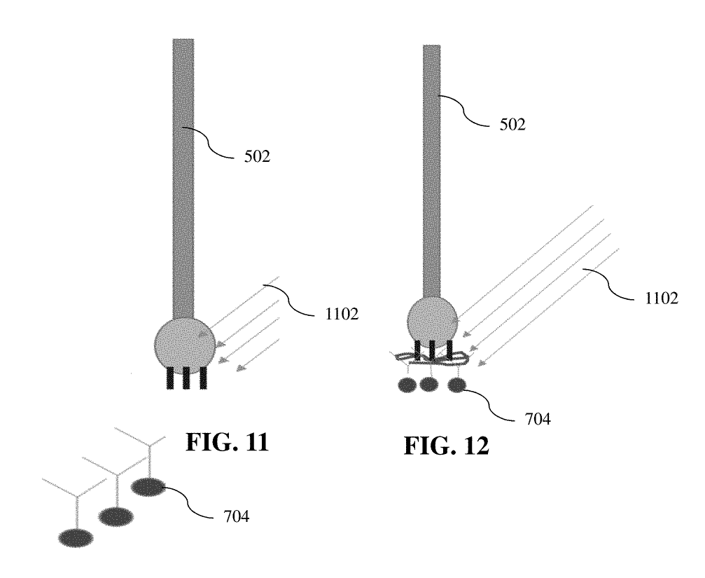

In step 104, the lyophilized antibody 704 will be reconstituted with about 3 mL (for example) of a liquid salt buffer at a specific salt concentration or a phosphate-buffered saline (PBS) 802, which is contained in the diagnostic kit (see FIG. 8). In step 106, the collection swab that has been used to collect the CSF and biomarker protein will be placed into the plastic tube 702 (see FIG. 9) containing the biomarker-specific antibody conjugated to the conjugates above and, in step 108, allowed to incubate for a set period of time, such as 15 minutes. During this time, the lyophilized biomarker-specific antibody 704 reacts with the biomarker 602 in the CSF on the swab 502 (see FIG. 10). Specifically, the lyophilized biomarker-specific antibody 704 binds to, or is coupled with, the biomarker 602 in the CSF.

After the incubation period, the swab 502 is removed from the tube in step 110. In step 112, the swab 502 may be rinsed thoroughly with distilled water 1102, which is contained in the diagnostic kit. If the diagnostic is negative for concussions, TBI, CTE, or other CNS afflictions, then the rinsing will remove the lyophilized antibody 704 and there will be no color change to the swab 502 (see FIG. 11). If the diagnostic is positive (meaning the biomarker is on the collection swab and has reacted with the biomarker-specific antibody), then the rinsing will not remove the lyophilized antibody 704 because the antibody-antigen interaction (or binding) will prevent the lyophilized antibody 704 from being washed away (see FIG. 12). In this case, in step 114 there will be a color change to the swab tip 504, indicating a positive test result for concussion. The color change is due to the color functionality of the conjugate, as described above.

If the diagnostic is negative for concussions, TBI, CTE, or other CNS afflictions, then there will be no color change to the swab 502. If the diagnostic is positive (meaning the biomarker is on the collection swab and has reacted with the biomarker-specific antibody), then in step 112 there will be a color change to the swab tip 504 indicating a positive test result for concussions, TBI, CTE, or other CNS afflictions. The color change is due to the color functionality of the conjugate, as described above.

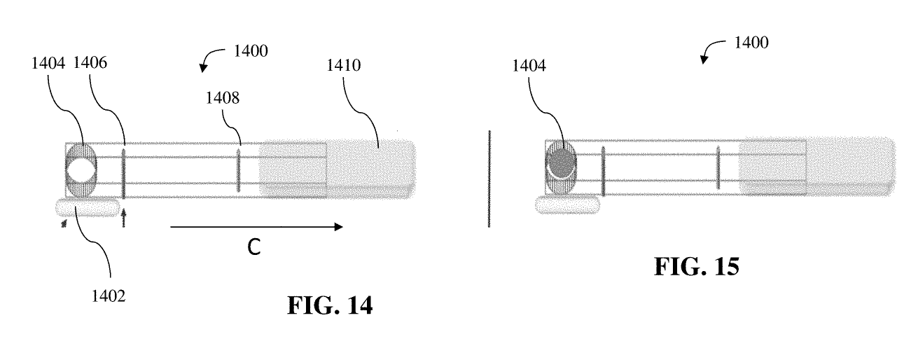

A second disclosed embodiment for diagnosing concussions, TBI, CTE, or other CNS afflictions, is disclosed below. This second detection method employs a rapid lateral flow capture assay (or test) 1400 (see FIG. 14) to detect the presence of a biomarker in discharged CSF, peripheral blood or CSF collected by lumbar puncture. Lateral flow tests, also known as lateral flow immunochromatographic assays, are simple devices intended to detect the presence (or absence) of a target analyte in sample (matrix) without the need for specialized and costly equipment. Typically, these tests are used for medical diagnostics either for home testing, point of care testing, or laboratory use. A widely spread and well known application is the home pregnancy test.

Vertical flow immunoassays rely on the same basic principles as the more frequently used lateral flow immunoassays with some modifications. The most obvious difference between the two methods being the vertical and lateral flow of sample fluid. However, there can be several advantages to the vertical flow assays as compared to the traditional lateral flow assays with the most significant being reduced time (less than 5 minutes).

As with the lateral flow, vertical flow immunoassays rely on the immobilization of a capture antibody on a pad to which the test sample (with or without antigen to be detected) is applied. Detection of the bound antigen is achieved through the binding of an antigen specific antibody conjugated to colored latex nanoparticles, or other conjugates listed above, such as colloidal gold. This step completes the “sandwich” composed of a capture antibody, and antigen and a conjugated antibody and results in a direct and visually detectable colored dot indicating the presence of the antigen. This technology, like lateral flow, also allows for multiplexing, where 4 different targets can be evaluated simultaneously in a single sample in 5 minutes or less.

Multiplexing is achieved by spotting capture antibodies against different antigens at pre-determined locations and or patterns on the membrane. For easier visualization; multiplexing can be performed with nanoparticles of different colors nanoparticles. For example, one antigen can be detected using red nanoparticles and the other antigen could be detected using blue nanoparticles.

The second disclosed method also uses a non-hydrophobic nylon-flocked nasopharyngeal diagnostic swab to capture discharged CSF from afflicted individuals. The swab may be equipped with a collar at 5.5 cm as a guide to maximum insertion depth. The swab should not be hydrophobic so that the biomarker protein is transferred to the nitrocellulose membrane and does not remain bound to the swab. Since a large volume of fluid is required for this second test, the flocked swab (see FIG. 4), which absorbs 3 times the fluid volume, is the preferred collection method for this second test. For ear collection, the second method uses a non-hydrophobic ear diagnostic collection swab, which is gently inserted into the ear and rotated to collect any discharged CSF. In addition, CSF collected by lumbar puncture can be applied to this diagnostic as well as peripheral blood collected by venipuncture can also be applied to this diagnostic in order to detect specific biomarkers.

The second method employs a specific IgG anti-concussion or CTE specific biomarker antibody at a specific concentration conjugated to an aforementioned conjugate along with the proper buffer conditions (salt concentration, pH, etc.) to facilitate antibody/antigen binding found applied directly to an antibody conjugate pad located just downstream of the sample application area (e.g., lateral flow test) (see item 1400 in FIG. 14). Downstream of the conjugated antibody conjugate pad there will be a second unconjugated antibody specific for the same biomarker as the conjugated antibody. This second unconjugated antibody will be applied directly to the nitrocellulose membrane binds where the unconjugated antibody proteins bind to the membrane electrostatically through interactions of the strong dipole of the nitrate ester with the strong dipole of the peptide bond of the protein, wherein the interaction is completely independent of the pH. In short, if the biomarker is present in sufficient concentration, the biomarker-specific antibody will bind to the biomarker in the CSF. Since the biomarker specific antibody has a color functionality attached to it, it will allow the visualization of the biomarker though a color change.

The overall total length of the membrane must meet certain criteria. If the nitrocellulose membrane is too long, sample diffusion will occur and hence decreased target sample concentration could prevent the detection of the biomarker. If the length of the nitrocellulose membrane is too short, it will have a negative effect on the resolution of the color indicator, making it blurry and uneven.

The second method may include antibodies that are affinity-purified, and employ a nitrocellulose membrane with a specific pore size and has a specific capillary flow rate. Further, the nitrocellulose membrane will have a specific overall total length.

Additionally, biomarkers can be used in combination in the disclosed diagnostic test assaying for potential severity of the concussion or a combination test for both concussion and CTE, or other CNS afflictions. For the combination assay with respect to using multiple biomarkers, any or all of the following markers can be used in any combination or all together in this embodiment (tau, S100B, NSE, UCH-L1, Non-erythroid αII-spectrin, Amyloid β, GFAP, and APP) as a potential way of diagnosing the severity of the concussion. To this end, antibodies specific against each concussion biomarker will be employed. Each of these antibodies will be conjugated to one or more of the aforementioned conjugates. The antibodies will be applied directly to the conjugate pad. The embodiment testing for multiple biomarkers works similarly as the embodiment testing for a single biomarker—the difference being the result will be multiple visual lines instead of one line and a control line.

The second method further employs a bed (see item 1410 in FIG. 14) for the filter paper of cellulose filter paper, binding buffers that are devoid of Tween 20 and Triton X-100 (both chaotropic agents that can physically interfere with the antibody protein and nitrocellulose interactions—thus, if these compounds are used, then they should be used at concentrations lower than 0.01% v/v), due the inhibitory binding activity of these compounds, and a specific membrane whose lateral flow rate is specific as a function of antibody-antigen complex formation where: R=k[AB] [Ag].

Capillary flow rate is important because the effective concentration of an analyte in the sample (the concussion and/or CTE-specific biomarker) is inversely proportional to the square of the change in flow rate. In a lateral flow or vertical flow test, the antigen is unable to bind once it passes the immobilized antibody because the test is designed to flow in only one direction. As a consequence of the test design the effective antigen concentration decreases with the square of the increase in flow rate because of the reduced length of time that the components of the reactive pair (antibody and antigen) are close enough to bind to each other. Further, doubling the flow rate effectively decreases the concentration of the complex by 4-fold which would make the detection of the complex formation very difficult: R=k[0.5×Ab] [0.5×Ag]=0.25 [Ab] [Ag]. The amount of antibody-antigen complex formed, R is equal to k, a rate constant related to the affinity of the antibody for the antigen, times the concentrations of the reactants AB (antibody) and AG (antigen). At a flow rate of 1×: R=k[AB][AG].

If the flow rate doubles, each component is only close enough for half the amount of time. [AB] and [AG] don't actually change; their effective concentrations change because they spend only half as much time in close proximity to bind each other.

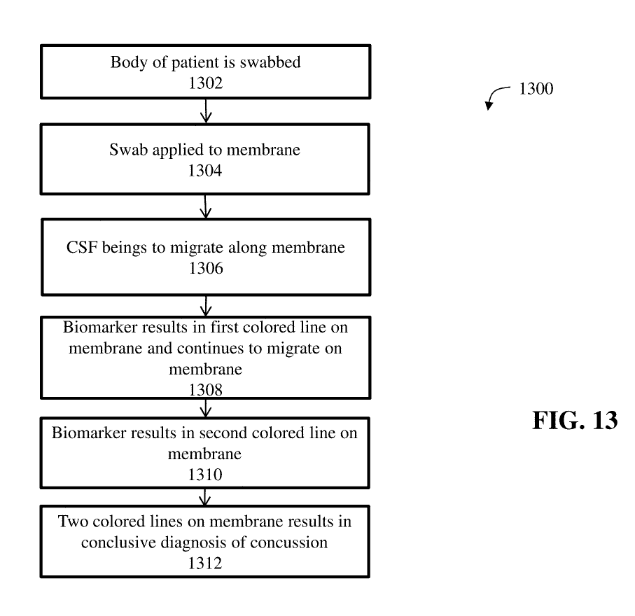

The process 1300 of FIG. 13 begins with a step 1302 wherein a portion of the body of the patient (ear canal, nasal cavity, etc.) is swabbed with the swab 502. Discharged CSF will be collected by the non-hydrophobic swab. This discharged CSF is the result of the insult to the head and will contain the specific biomarker. In the event that one does not perform swabbing of the nasal passage or ear canal within an hour post-injury, a spray bottle containing sterile saline is included in the kit. Slight irrigation of nose or ear will facilitate capture of the biomarker on the swab and facilitate optimal transfer to the membrane.

In step 1304, the fluid on the swab will then be transferred onto the sample application area 1404 (see FIG. 14) of the diagnostic nitrocellulose membrane, such as via sample pad 1402. If the amount of CSF fluid is small, a small squirt bottle will be available to mix the CSF fluid into and that fluid (Tris buffer solution, for example) will be used to apply the CSF directly onto the sample application area 1404 of the concussion diagnostic membrane, or sample pad 1402.

The diagnostic system will already contain the biomarker antibody conjugated to a conjugate already applied and dried on the conjugate pad component of the diagnostic system. This antibody will be applied to the conjugate pad under specific buffer and pH conditions and without the use of chaotrophic agents, which could potentially physically disrupt the interactions of the antibody to the membrane.

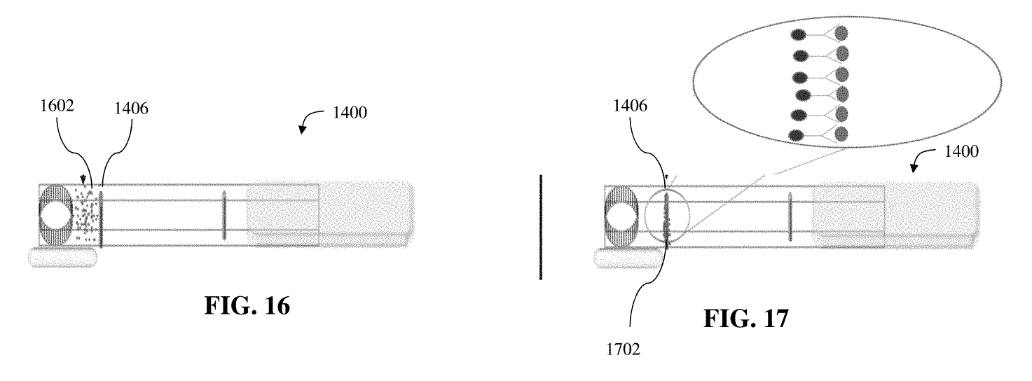

After the application of the CSF to the sample application area 1404 of the diagnostic membrane, in step 1306, the CSF fluid will begin to migrate via lateral flow (capillary action—direction of arrow C in FIG. 14) down the membrane and through the conjugate pad region where the conjugated antibody will bind to the specific biomarker. After passing through the conjugate pad and subsequent antibody-biomarker binding, the complex continues migrating through the membrane downstream from the conjugate pad. FIG. 16 shows the CSF 1602 travelling towards 1406. The target capture line 1406 consists of a biomarker-specific antibody conjugated to a conjugate.

Once the biomarker comes into contact with the target line 1406, in step 1308, the biomarker-specific antibody conjugated to the conjugate will bind to the biomarker and continue migrating with the protein down the membrane. The biomarker binds to the conjugate, and the conjugated antibody carries the antibody along with it during lateral flow or vertical flow. Not all of the conjugated antibody will migrate with the concussion and or specific biomarker, however, leaving some behind, and therefore leaving an initial colored line at 1406.

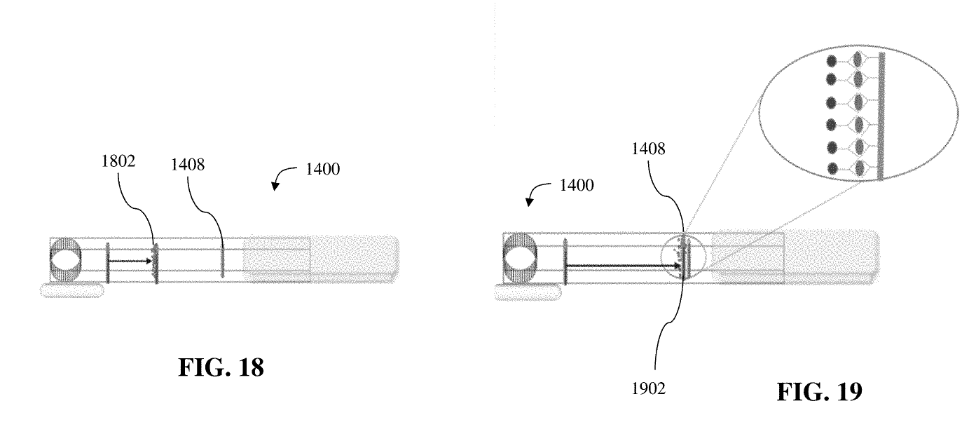

FIG. 17 shows that the CSF 1702 has reached line 1406. If biomarker is present, it will bind irreversibly to the anti-biomarker antibody conjugated to the conjugate and form a biomarker-antibody-conjugate complex at line 1406. FIG. 18 shows that the biomarker and anti-biomarker conjugated antibody 1802 has passed line 1406 and continues to migrate through the nitrocellulose membrane towards 2^{nd} line 1408.

The specific biomarker-conjugated antibody will migrate until it comes in contact with the second capture line 1408. The capture line 1408 also consists of a second specific antibody, IgG, which is not conjugated and binds to a different epitope of the specific biomarker. Line 1408 will “capture” the initial specific biomarker-anti-biomarker conjugated antibody and create a 2^{nd} colored line on the diagnostic membrane in step 1310. The specific biomarker conjugated antibody is then captured by the second unconjugated specific biomarker antibody immobilized on the membrane. FIG. 19 shows that the CSF 1902 has reached line 1408.

If the specific biomarker is present, which will be the indicator of this diagnostic, it will be captured by the unconjugated antibody 1408 and the result will be the presence of a 2nd colored line on the nitrocellulose membrane at 1408. If the biomarker is not present, there will not be the formation of a second colored line at 1408 thereby yielding a negative result on the diagnostic. Thus, the presence of the specific biomarker will result in the presence of at least two colored lines 1406 and 1408, but the lack of the specific biomarker will not result in two colored lines. Consequently, at least two colored lines 1406 and 1408 in the diagnostic membrane results in a conclusive diagnosis of a concussion, TBI, CTE, MS, ALS, etc. in step 1312.

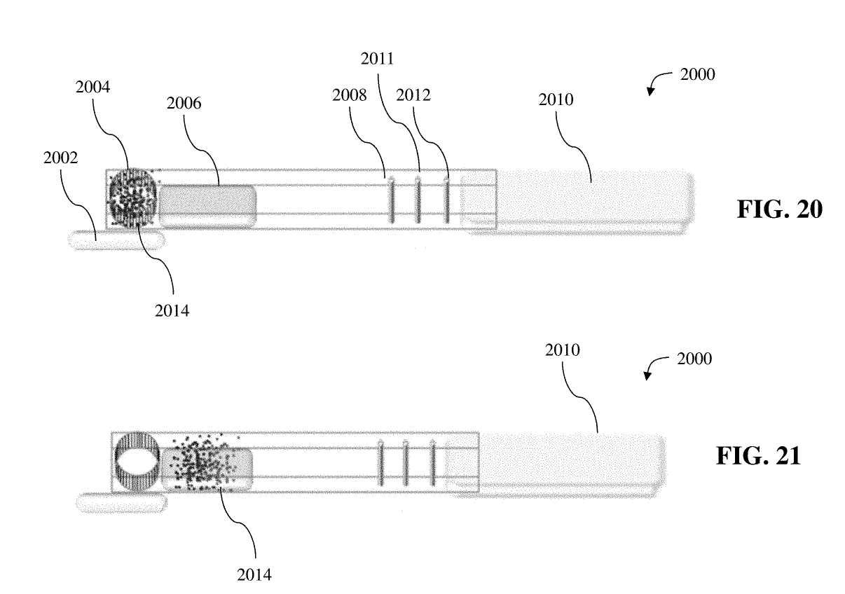

FIGS. 20, 21, 22, 23 are illustrations of a second embodiment of a nitrocellulose membrane in various stages of the process for diagnosing concussions, TBI, CTE, ALS, MS, meningitis and other CNS afflictions, according to one embodiment. Aforesaid figures show a rapid lateral flow capture assay (or test) 2000 with a sample application area 2014 (see FIG. 20) of the diagnostic nitrocellulose membrane, to which the sample is applied via sample pad 2002. The sample moves toward the capture pad 2006 via capillary action, and subsequently to capture lines 2008, 2011 and 2012. Eventually, the sample ends is movement when it reaches the absorbent pad 2010.

The second embodiment may include antibodies at a first capture line 2008 that are affinity-purified, and employ a nitrocellulose membrane with a specific pore size and has a specific capillary flow rate. Further, the nitrocellulose membrane will have a specific overall total length. The second embodiment may also employ a 2nd unconjugated antibody that has been applied directly to the nitrocellulose membrane (at 2^{nd} capture line 2011) downstream from the 1st anti-concussion or CTE biomarker antibody (at the first capture line 2008) and functions as the CTE test line. In addition, there is a 3rd capture line 2012 on the membrane that consists of an antibody directed against the conjugated antibody that serves as the control line. The control line 2012 antibody is specific for the conjugated biomarker antibody and this control line functions to demonstrate that the lateral flow or vertical flow test is working, i.e. this system has been designed to permit the lateral flow of antibodies and proteins through the membrane, this means that the conjugated biomarker antibody will migrate through the membrane whether or not the specific biomarker is present. The control line functions to capture this conjugated antibody whether or not it is bound to the specific antigen and this is designed to result in the presence of a single line. The control line includes antibodies configured for binding to the anti-biomarker antibodies (not to the biomarker). This control line shows that the system is working and the proteins are flowing through system.

A sample may be transferred onto the sample application area 2004 (see FIG. 14) of the diagnostic nitrocellulose membrane, such as via sample pad 2014. If the amount of CSF fluid is small, a small squirt bottle will be available to mix the CSF fluid into and that fluid (Tris buffer solution, for example) will be used to apply the CSF directly onto the sample application area 2004. The CSF fluid 2014 will begin to migrate via lateral flow down the membrane and through the conjugate pad region 2006 where the conjugated antibody will bind to the specific biomarker. After passing through the conjugate pad and subsequent antibody-biomarker binding, the complex 2014 continues migrating through the membrane towards the capture lines 2008, 2011 and the control line 2012 downstream from the conjugate pad. FIG. 21 shows the CSF 2014 travelling towards 2008. The target capture line 2008 consists of a biomarker-specific antibody conjugated to a conjugate. Once the biomarker comes into contact with the capture lines, the biomarker-specific antibody conjugated to the conjugate will bind to the biomarker and continue migrating with the protein down the membrane. The biomarker binds to the conjugate, and the conjugated antibody carries the antibody along with it during lateral flow or vertical flow. Not all of the conjugated antibody will migrate with the concussion and or specific biomarker, however, leaving some behind, and therefore leaving an initial colored line at 1406.

The specific biomarker-conjugated antibody will migrate until it comes in contact with the second capture line 2011. The capture line 2011 also consists of a second specific antibody, IgG, which is not conjugated and binds to a different epitope of the specific biomarker. Line 2011 will “capture” the initial specific biomarker-anti-biomarker conjugated antibody and create a 2nd colored line on the diagnostic membrane. The specific biomarker conjugated antibody is then captured by the second unconjugated specific biomarker antibody immobilized on the membrane.

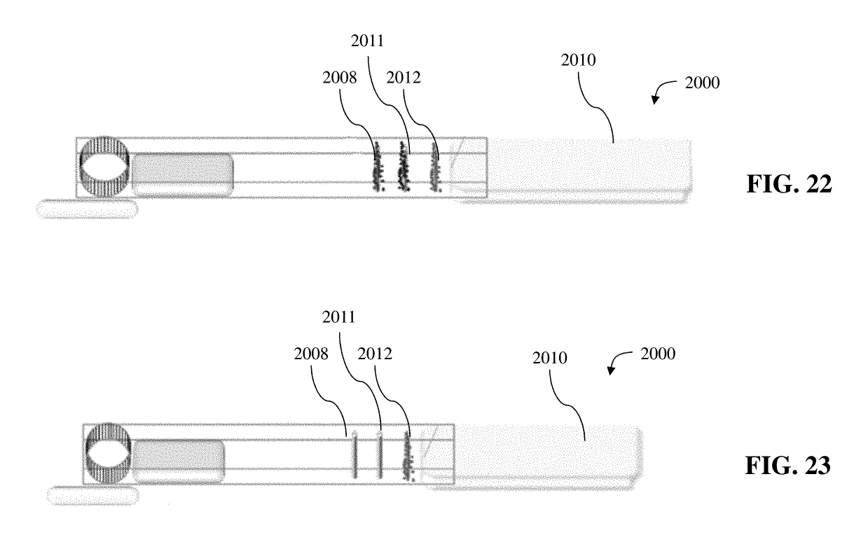

If the specific biomarker is present, which will be the indicator of this diagnostic, it will be captured by the unconjugated antibody and the result will be the presence of a 2nd colored line on the nitrocellulose membrane at 2011. If the biomarker is not present, there will not be the formation of a second colored line at 2011 thereby yielding a negative result on the diagnostic. Thus, the presence of the specific biomarker will result in the presence of at least two colored lines 2008 and 2011, but the lack of the specific biomarker will not result in two colored lines. Recall the control line 2012, which will result in a colored line 2012 if the CSF fluid 2104 reaches the control line. Consequently, the colored lines 2008, 2011, 2012 in the diagnostic membrane results in a conclusive diagnosis of a concussion, TBI, CTE, MS, ALS, etc. FIG. 22 shows that the CSF 2014 has reached (and changed the color of) capture lines 2008, 2011, as well as control line 2012, which is a positive diagnostic test. FIG. 23 shows that capture lines 2008, 2011 are not colored, but that control line 2012 is colored, which is a negative diagnostic test, and a confirmation that the test has worked, since the control line changed color.

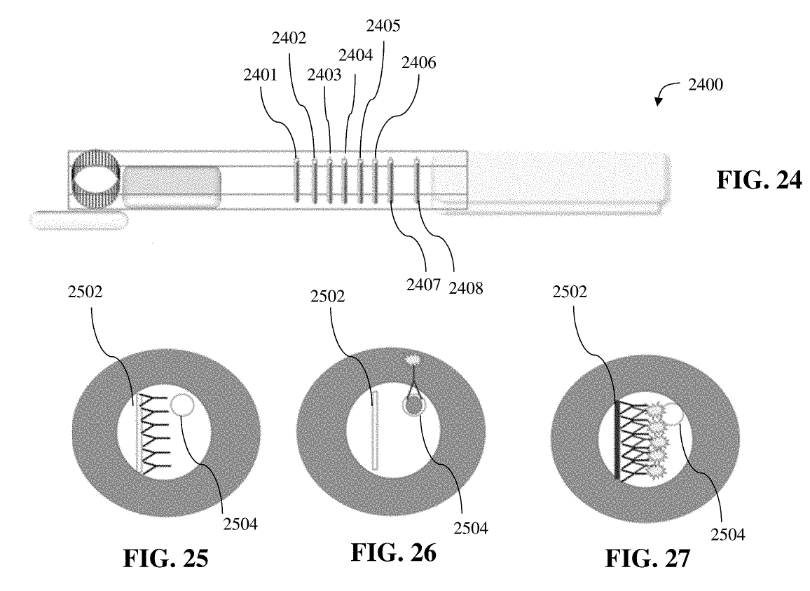

FIG. 24 is an illustration of a third embodiment of a nitrocellulose membrane for diagnosing concussions, TBI, CTE, ALS, MS, meningitis and other CNS afflictions, according to one embodiment. FIG. 24 shows that seven different unconjugated antibodies specific for concussion and/or CTE-specific biomarkers can be applied on seven different capture lines (2401 through 2407) on one test (see item 2400). Finally, said test can also include a control line 2408.

FIGS. 25, 26, 27 are illustrations of a fourth embodiment of a nitrocellulose membrane in various stages of the process for diagnosing concussions, TBI, CTE, ALS, MS, meningitis and other CNS afflictions, according to one embodiment. FIGS. 25, 26, 27 pertain to a vertical flow immunoassay, which, recall, relies on the same basic principles as the more frequently used lateral flow immunoassays with some modifications. There can be several advantages to the vertical flow assays as compared to the traditional lateral flow assays with the most significant being reduced time. FIG. 25 shows an embodiment including a control line 2502, and a capture dot 2504 (including unconjugated antibodies specific for concussion and/or CTE-specific biomarkers). FIG. 26 shows that the capture dot has changed color, indicating the presence of a biomarker. FIG. 27 shows that the capture dot has not changed color, indicating the absence of a biomarker, and the control line 2502 has changed color, indicating the test has worked properly.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A diagnostic kit for diagnosing concussions, traumatic brain injury, chronic traumatic encephalopathy and other central nervous system disorders, the diagnostic kit comprising:
a tube containing a predefined amount of lyophilized biomarker-specific antibody conjugated to a conjugate with a color functionality;
a fluid for mixing with the lyophilized biomarker-specific antibody conjugated to the conjugate within the tube, so as to reconstitute the lyophilized biomarker-specific antibody conjugated to the conjugate, resulting in a reconstituted mixture; and
a swab comprising an absorbent hydrophobic material, the swab configured for absorbing cerebrospinal fluid when swabbed on a patient, wherein when the swab that has absorbed cerebrospinal fluid is placed in the tube containing the reconstituted mixture, the swab is configured to change color.

2. The diagnostic kit of claim 1, wherein the swab includes a swab tip configured for binding to hydrophobic amino acids in said biomarker in cerebrospinal fluid of the patient.

3. The diagnostic kit of claim 2, wherein the lyophilized biomarker-specific antibody in the tube comprises about 0.5 mg of lyophilized biomarker-specific antibody.

4. The diagnostic kit of claim 3, wherein the conjugate comprises colloidal gold.

5. The diagnostic kit of claim 4, wherein the fluid for mixing with the lyophilized biomarker-specific antibody within the tube comprises about 3 ml of a predefined buffer at a specific pH and salt concentration.

6. A method for diagnosing concussions, traumatic brain injury, chronic traumatic encephalopathy and other central nervous system disorders, the method comprising:
swabbing a patient with a swab comprising an absorbent hydrophobic material, so as to absorb cerebrospinal fluid of the patient into the swab;
pouring a fluid into a tube containing a predefined amount of lyophilized biomarker-specific antibody conjugated to a conjugate with a color functionality, so as to reconstitute the lyophilized biomarker-specific antibody conjugated to the conjugate, resulting in a reconstituted mixture;
placing the swab in the tube containing the reconstituted mixture for a predefined period of time;
removing the swab from the tube and rinsing with water; and
observing a change of color of the swab, wherein the swab is configured to change color when cerebrospinal fluid of the patient is absorbed into the swab.

7. The method of claim 6, wherein the step of swabbing a patient further comprises:
swabbing a patient with a swab comprising an absorbent hydrophobic material, so as to absorb cerebrospinal fluid of the patient into the swab, wherein the swab includes a swab tip configured for binding to hydrophobic amino acids in said biomarker in cerebrospinal fluid of the patient.

8. The method of claim 7, wherein said predefined amount of lyophilized biomarker-specific antibody comprises about 0.5 mg of lyophilized biomarker-specific antibody.

9. The method of claim 8, wherein said conjugate comprises colloidal gold.

10. The method of claim 9, wherein said fluid comprises about 3 ml of a predefined buffer at a specific pH and a specific salt concentration.

11. The method of claim 10, wherein said predefined period of time comprises about 15 minutes.

12. A diagnostic kit for diagnosing concussions, traumatic brain injury, chronic traumatic encephalopathy and other central nervous system disorders, the diagnostic kit comprising:
a swab comprising an absorbent swab, the swab configured for absorbing cerebrospinal fluid when swabbed on a patient;
a high flow nitrocellulose membrane configured for lateral flow in a first direction via capillary action;
a sample loading area located at a first end of the membrane and upstream of the first direction;
a first set of anti-biomarker antibodies, conjugated to a conjugate with color functionality, located at a first location on the membrane and downstream of the sample loading area;
a second set of anti-biomarker unconjugated antibodies located at a second location on the membrane and downstream of the first location; and
a cellulose absorbent cloth located at a second end of the membrane and downstream of the first location, so as to aid the lateral flow;
wherein when the sample loading area has absorbed cerebrospinal fluid transferred from the swab, and the cerebrospinal fluid flows laterally in the first direction, the first and second locations on the membrane are configured to change color.

13. The diagnostic kit of claim 12, wherein the swab includes a flocked swab tip.

14. The diagnostic kit of claim 13, wherein the conjugate comprises colored latex nanoparticles, including red latex nanoparticles.

15. The diagnostic kit of claim 14, wherein the colored latex nanoparticles comprise red latex beads.

16. The diagnostic kit of claim 13, wherein the first set of antibodies comprise IgG antibodies.

17. The diagnostic kit of claim 13, wherein the second set of antibodies comprise IgG antibodies.

18. The diagnostic kit of claim 12, wherein anti-biomarker antibodies comprise antibodies that are configured to bind to the Tau protein.

19. The diagnostic kit of claim 12, wherein anti-biomarker antibodies comprise antibodies that are configured to bind to a biomarkers selected from the group consisting of: Tau, S100B, NSE, UCH-L1, Non-Erythroid alpha II spectrin, Amyloid Beta, GFAP, APP, Phosphorylated Tau, and TDP-43.

20. The diagnostic kit of claim 12, wherein the conjugate comprises colloidal gold.

21. The diagnostic kit of claim 12, wherein the conjugate comprises a substance selected from the group consisting of: colored latex nanoparticles, colloidal gold, colloidal silver, quantum dots, fluorescent latex particles, up-converting phosphors, enzymes, colloidal carbon, colloidal platinum, lipsome-based probes, magnetic particles, and Raman-active tags.

22. The diagnostic kit of claim 12, wherein the swab includes a non-hydrophobic flocked swab.

23. The diagnostic kit of claim 12, wherein the other central nervous system disorders comprise a disorder selected from the group consisting of: multiple sclerosis, amyotrophic lateral sclerosis, and meningitis.
